(19) United States
(12) Patent Application Publication
(10) Pub. No.: US 2013/0018445 A1
(43) Pub. Date: Jan. 17, 2013

(54) NEUROSTIMULATION LEAD

(71) Applicants: JONATHAN L SAKAI, Fairview Park, OH (US); Joseph W. Boggs, II, Carrboro, NC (US)

(72) Inventors: JONATHAN L SAKAI, Fairview Park, OH (US); Joseph W. Boggs, II, Carrboro, NC (US)

(73) Assignee: NDI MEDICAL, LLC, CLEVELAND, OH (US)

(21) Appl. No.: 13/351,953

(22) Filed: Jan. 17, 2012

Related U.S. Application Data
(60) Provisional application No. 61/433,027, filed on Jan. 14, 2011.

(51) Int. Cl. A61N 1/05

(52) U.S. Cl. 607/116

(57) ABSTRACT

Improved electrical stimulation leads are disclosed. Such leads may be provided with a distal electrode and electrode retention elements, such as tissue anchors. The electrode may be electrically coupled to an electrical connector that is disposed at a proximal end of the lead through one or more cables, which may conduct electricity through one or more conductive strands. The electrode may be formed from an extension of one of such cables coiled about a portion of the lead body.

### RELATED APPLICATIONS

This application claims the benefit of U.S. Provisional Patent Application Ser. No. 61/433,027 filed 14 Jan. 2011, and entitled “Neurostimulation Lead,” which is incorporated by reference herein in its entirety.

### BACKGROUND OF THE INVENTION

Systems and methods according to the present invention relate generally to the field of electrical stimulation delivery and more specifically to improved embodiments of electrical stimulation leads and/or electrodes.

Presently available neurostimulation leads are not generaUy suited for implantation In and sustained. operation in or adjacent to large peripheral muscles. Common lead designs presently used by many researchers were developed for therapies utilized by persons with limited mobility and activity. Research into the durability of prior designs has generally been limited. to leads utilized in a neuroprosthesis to restore upper-limb function, meaning the lead is used in joints and muscles that see a low level of activity (reduced forces, range of motion and number of cycles) compared to asymptemic persons. A more reliable, robust neurostimulation pain relief system may be rewired in certain circumstances, such as may be required to treat athletes, military personnel, or other active individuals. To accommodate such activity, if a neurostimulation system will utilize a wired lead, such stimlation lead needs to demonstrate an extreme level of durability in and around major muscle groups that will be subjected to higher levels of stress and higher cycle counts than experienced by present intramuscular leads used in civilian. therapies.

While generally a stimulation lead according to the present invention may be used in a variety of stimulation systems and methods, such as the treatment of neuropathic pain, one application of an improved. electrical stimulation lead according to the present invention may be a novel non-narcotic, non-addictive, easily-deployable robust medical device to treat severe and disabling combat-trauma pain for military personnel. Over 30,000 US service members have been injured since the start of Operation Enduring Freedom/Operation Iraqi Freedom, and over 95% of soldiers with combat-trauma suffer from pain. A method utilizing a stimulation lead according to the present invention may deliver therapeutic stimulation through a lead that can withstand. the extreme and repeated stresses of rigorous exercise. Such treatment may provide pain relief without narcotics and allow tens of thousands of soldiers to return to active duty.

Physicians that are pain specialists, such as anesthesiologists, typically treat severe pain with medications (primarily narcotics) that carry the risk of addiction, often fail, to alleviate the pain, and lead to unwanted side effects such as nausea, confusion, vomiting, hallucinations, drowsiness, dizziness, headache, agitation, and insomnia. Other medications (such as antidepressants and anticonvulsants), physical therapy, and psychological methods are often tried but are seldom successful in controlling the pain. None of them is consistently successful and all of them are ultimately insufficient.

Although a few case studios have suggested.

that electrical stimulation of the injured nerve could relieve severe pain, it is rarely attempted because no one has developed an easily-deployable and robust electrode that can reliably deliver stimulation to peripheral nerves given rho extreme stresses experienced by electrodes placed in or around large muscle groups. The complex, invasive surgery required to glace the existing electrodes on the delicate connective tissue surrounding the injured nerve is too risky and complicated to be attempted in most patients. In the few cases where electrodes have been placed, they often fracture or are dislodged, leading to loss of pain relief and revision surgeries.

Accordingly, there remains room in the neurostimulation art to simplify electrode placement and greatly minimize the risk of tissue injury in active persons.

### SUMMARY OF THE INVENTION

Embodiments according to the present invention may include an implantable neurostimulation lead designed to be implanted in muscle or adipose tissue and to be sufficiently rugged to withstand use by highly active persons such as athletes or military personnel.

This lead may be used in conjunction with neurostimulation devices, implanted or external, that require routing an electrical impulse between a device and an electrode across a joint that will be subjected to high amounts of stress and cyclic movement. Peripheral pain management therapies that will allow persons to return to high levels of activity will require a highly durable lead, as will devices that will be used by military personnel. The lead design provides an as-yet unmet need; a rugged neurostimulation lead that can be used in adipose tissue or muscle.

According to one aspect of an embodiment of a stimulation lead according to the present invention, lead retention barbs/tines may be included to maintain an electrode position in adipose tissue, muscle tissue, or a combination of such tissues, or other animal body tissue.

According to another aspect of an embodiment of a stimulation lead according to the present invention, one or more electrodes may be electrically coupled to an electrical connector though one or more highly stranded cables, such as cables including 7, 19, or even 49 conductive strands. Such stranded cables may be individually insulated with flouropolymer.

According to yet another aspect of an embodiment of a stimulation lead according to the present invention, multiple cables may be coupled in parallel between one electrode and a connector element may be provided for redundancy and reduction in resistance. The cable (s) may be surrounded by a flexible jacket, such as a silicone tubing jacket.

According to still another aspect of a stimulation lead according to the present invention, cables that electrically couple an electrode to an electrical connector may be coiled about an open lumen formed by the coiled cables. Accordingly, a stylet may be placed in the lead initially to facilitate placement.

According to a further aspect of a stimulation lead according to the present invention, an electrode may be formed from a wound coil construction composed of the same cable, or strand thereof, that is used to electrically couple the electrode to an electrical connector.

### BRIEF DESCRIPTION OF THE DRAWINGS

FIG. 1 is a perspective view of an embodiment of an electrical stimulation lead incorporating elements according to the present invention,

FIG. 2 is a close-up perspective view of a first portion of the embodiment of FIG. 1.

FIG. 3A is a perspective partial cutaway view of the embodiment of FIG. 1,

FIG. 3B is a close-up perspective cutaway view of a second portion of the embodiment of FIG. 1.

FIG. 3C is a close-up cross-section view of a portion of FIG. 3B.

### DESCRIPTION OF THE PREFERRED EMBODIMENT

Although the disclosure hereof is detailed and exact to enable those skilled in the art to practice the invention, the physical embodiments herein disclosed merely exemplify the invention. which may be embodied in other specific structures. While the preferred embodiment has been described, the details may be changed without departing from the invention, which is defined by the claims.

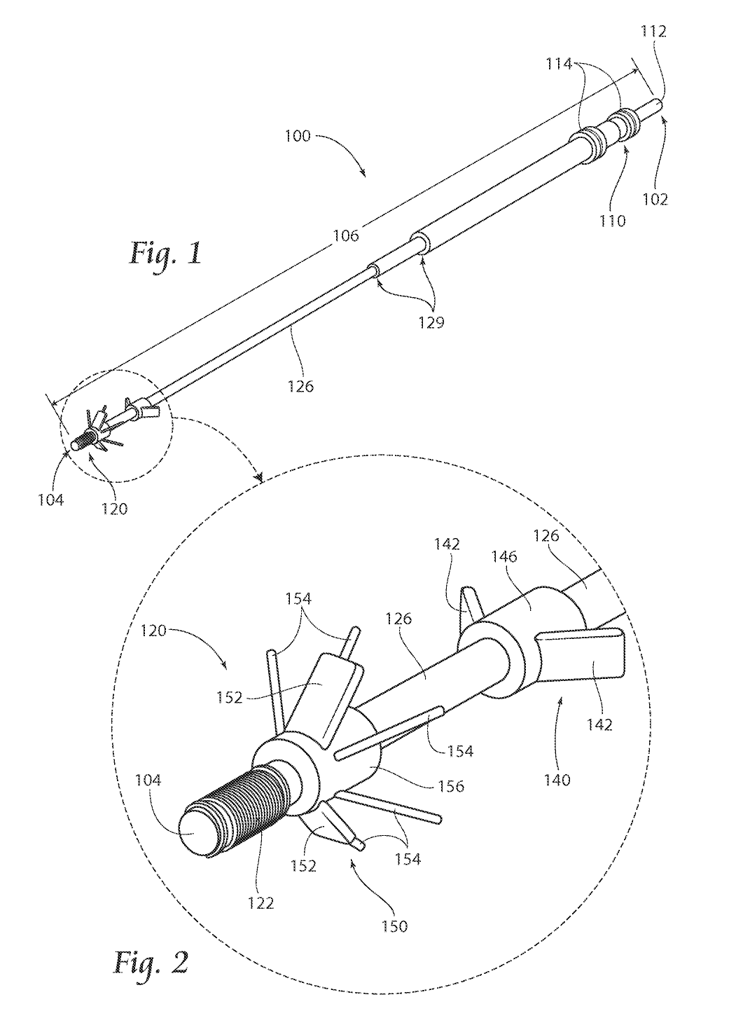

Turning now to the figures, FIG. 1 depicts a preferred embodiment 100 of a stimulation lead. The lead 100 extends between a proximal end 102 and a distal end 104, along a lead. length. 106. A preferred lead length 106 is between 5 and 100 centimeters, and more preferably about 30 to about 100 centimeters. Provided at the proximal end 102 is an electrical connector element 110. The connector element 110 may be any construction required to mate to a stimulation pulse generator, such as an implantable pulse generator. An IS-1 type connector, as is known, may be used, for example. The connector 110 preferably includes an electrical conductor 112 and may further include jack sealing members 114 adapted to interface with a pulse generator and preferably fluid imperviously seal the electrical coupling of the conductor 112 with a pulse generator conductor (not shown).

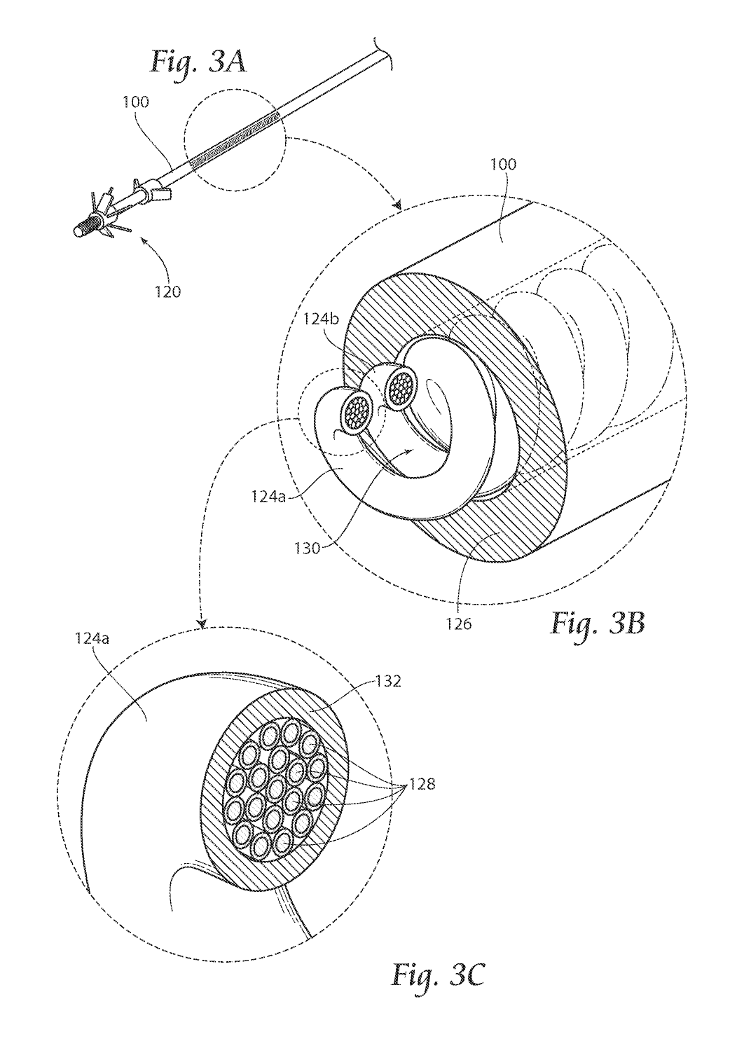

Provided at or near the distal end 104 of the lead 100 is an electrode arrangement 120. The arrangement 120 includes at least one electrode 122, which may be of a wound coil construction. The electrode 122 is electrically coupled to the electrical conductor 112 of the connector element 110. Such coupling may be provided by one or more highly stranded (e.g. 19-strand, 49-strand (7×7), or other highly stranded cable configuration) cables 124, preferably disposed in parallel. Each cable 124 is preferably helically coiled and placed within a flexible silicone jacket 126. The jacket 126 may include one or more diameter changes 129, which may be gradual changes, or step changes as shown. If diameter changes 129 are included, the diameter of the jacket 126 preferably decreases in the distal direction. The exact cable configuration (number of strands 128) may depend on a balance between desired resistance, flexibility, and the maximum size of the lead. Each strand 128 preferably includes an approximately 0.001″ diameter metallic wire, such as 316L stainless steel, (Other biocompatible metals suitable for delivering long-term stimulation may be used.) The present design preferably includes strands 128 having a construction of stainless steel, drawn-filed tube containing approximately 33% silver by cross sectional area. Using drawn filled tubing preferably provides a lower resistance (approximately 10 ohms for a 40 cm lead compared to 60 ohms for a 40 cm lead composed of solid. stainless steel wire). The lead 100 is preferably hollow along a majority of its length 106, thereby providing a lumen 130, which may be configured to accommodate a rigid stylet, which will aid in lead placement and removed at the end of implantation. The lumen 130 is preferably provided through at least a portion of the connector element 110, too. The lead electrode 122 may be a helically wound coil composed of one or more of the same 316L stainless steel wires used for the strands 128, The silver core of the strands 128 is preferably not exposed to prevent tissue contact with same. The lead electrode 122 is preferably the distal--most functional aspect of the device, and may allow the surgeon to deliver test simulation with the exact lead that may be deployed into the tissue. FIG. 3B provides a section view showing lead cable construction. Two cables, preferably at least substantially identical, colored light/dark to illustrate that these are individual cables. Bott cables could be electrically tied together for redundancy and decrease in resistance, or used for separate electrode contacts.

To form the coiled wire electrode 122, a strand 128 or cable 124 may be cut to a length that is much longer than the desired finished lead length 106, This strand 128 may then be straightened and, if provided, a preferred flouropolymer insulation 132 may be removed mechanically or using “hot tweezers”. This deinsulated strand 128 may then be wound back onto the distal end of the insulative silicone tubing 126, thereby forming a tight coil electrode 122. Any excess strand 128 may be trimmed and/or threaded into the lead body (or the anchoring tines) and sealed with adhesive. This electrode design preferably is configured to be flexible in construction so that it can move and deform with bodily tissue once implanted, particularly with muscle tissue. Other reasons that a strand 128 of the a cable 124 is preferably used to form the electrode 122 is to prevent having to do a welding, soldering or crimping operation to connect a rigid, solid ring electrode. While not impossible to be employed in combination with other aspects of the present invention, artificial joints present further opportunity for failure in-vivo.

A lead according to the present invention preferably features one or more retention mechanisms. For instance, a lead 100 may include one or more tine sets designed to retain lead position in either adipose or muscle tissue. The retention mechanisms may include a proximal tine set 140 and/or a distal tine set 150. The distal tine set 150 preferably features a combination of a plurality of elastomeric (e.g., polyurethane) “paddle” tines 152 and a plurality of elongate tines 154, which are preferably more rigid than the paddle tines 152. A preferred material for the elongate tines 154 may be, e.g., polypropylene. While the proximal tine set 140 may include both elongate and paddle type tines, the proximal tine set 140 preferably consists of only elastomeric “paddle” tines 142. The proximal tines 142 are preferably coupled to a hollow, cylindrical. proximal tine collar 146, which can he adhered or otherwise fixed to, or formed integrally with, the silicone jacket 126. The tines 142 are preferably spaced equally around the collar 146. For example, if two paddle tines 142 are provided, they are preferably spaced 180 degrees apart; if three tines 142 are provided, they are preferably spaced at 120 degrees. The distal tines 152,154 are preferably coupled to a hollow, cylindrical distal tine collar 156, which can be adhered or otherwise fixed to, or formed integrally with, the silicone jacket 126. The distal tine collar 156 may be formed integrally with one or more of the tines 152,154. The distal tine set 150 preferably includes a plurality, e.g., six, polypropylene barbs 154, which may be overmolded with two elastomeric paddle times 152. If both a proximal tine set 140 and a distal tine set 150 are provided on a lead 100, and if both sets 140,150 have paddle tines 142,152, the respective paddle tines are preferably not aligned along the length 106 of the lead 100. For example, in FIG. 2, the proximal paddle tines 142 are depicted as being 90 degrees out of phase with the distal tines 152.

Embodiment of stimulation leads according to the present invention provide a novel substantially permanently-implantable electrode with the mechanical strength to endure the stresses of rigorous exercise and preferably remain operational during 20 years of active military duty or other activity. The leads strive to eliminate what is conventionally a site of frequent fracture at the junction of the conductor wire and the contact surface by replacing the traditionally rigid. metal cylinder with a flexible spring-like surface made from one or more continuous biocompatible stainless steel conductor cables 124, or strands 128 thereof. The dual helically-coiled cables help to provide redundancy and increased mechanical strength. Dislodgement will be prevented in both muscle and adipose tissue with retention elements, which may include a plurality of types of anchoring tines. Preferred lead resistance does not exceed 1.0 ohms/cm.

Stimulation leads according to the present invention preferably use only a single electrode contact surface 122, directing focus towards reliability, redundancy, and resistance to extreme and repeated. mechanical stresses. The lead contains preferably two (redundant) cables 124 helically wound and enclosed inside silicone tubing 126 for increased flexibility and fatigue resistance. Twisting 19 thin stainless steel drawn-filled tubes 128 of 33% silver together reinforces the mechanical resistance to crush forces, decreases the electrical impedance, and provides additional redundancy. Creating a single contact surface 122 by coiling continuous strands 128 of the stainless steel from the deinsulated cables reduces the potential for failure by adding the flexibility of a spring-like coil instead of a traditional rigid metal cylinder and eliminating what is conventionally a site of frequent fracture between two disparate metals. Unwanted dislodgment of the electrode 122 in use will be reduced by the retention elements, which may include 6 rigid polypropylene tines that will anchor the electrode in muscle tissue and the 2 pairs of paddle tines that will anchor the electrode in adipose tissue.

The 2×19 coiled cable construction helps resist axial fatigue and can be reversibly stretched to over 160% elongation by uncoiling before the wire is straightened, decreasing the tensile forces imparted on the electrode and providing a safety factor greater than 6 times the anticipated 25% stretching in the leg or arm. The silicone tubing 126 chosen for the electrode preferably is able to elongate to over 800% of its resting length before failure.

During bending and torsion, a straight wire translates the energy into compression and tension stress, which can lead to failure, but lead embodiments according to the present invention that use a. helical coil construction reduce stress by distributing the majority of the energy into bending, twisting and deflecting of the coiled assembly and limiting the compression and tension stress imparted to the wires. The open coil construction (an air gap 130 within the lead) allows the coils to flex and reversibly deform under bending and torsion without contacting adjacent rigid structures.

Resilience to crushing may be provided by the highly elastic outer silicone tube 126 that assists in evenly distributing applied external forces across the coiled cables 124. When the distributed crushing force is transmitted to the cables 124, the coiled configuration receives the forces by deforming (causing the cables 124 to lay down in the direction of the coil) and passing those forces to the soft (muscle or adipose) tissue. In practice, the hollow lumen 130 will allow the device to be compressed fully under a crushing load and further distribute the load to adjacent tissue. The hollow core serves an additional function of accommodating a rigid stylet (not shown), which will aid in electrode placement and be removed following implantation.

Embodiments of stimulation leads according to the present invention are intended to survive and continue to function within specifications in the event of multiple failures in multiple locations. Each cable strand 128 is designed with an outer tube of stainless steel to maximize strength and an inner tube of silver to maximize conductivity, which increases the redundancy of each additional wire, Each cable uses 19 twisted strands to provide 18-fold redundancy in parallel conductors, meaning that up to 18 strands can fracture due to any stress at any single location without producing a significant change in overall conductivity of the cable. Furthermore, even if all 19 strands fracture at the exact same location in one cable, the second cable, if used, will still continue to provide sufficient conductivity. Additionally, the second cable also will be able to withstand fracture or up to 18 wires in any location and still provide sufficient conductivity to operate within specifications.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A neurostimulation lead comprising:
a lead body extending along a lead length from a proximal end to a distal end;
at least one lead retention element extending radially outward from said lead body;
a connector element disposed at said proximal end, said connector element comprising an electrically conductive surface;
an electrode disposed closer to said distal end than all of said at least one lead retention element;
wherein said electrode is electrically coupled to said electrically conductive surface.

2. A neurostimulation lead according to claim 1, wherein the electrode is electrically coupled to said electrically conductive surface through a plurality of insulated, electrically conductive cables.

3. A neurostimulation lead according to claim 2, wherein at least one electrically conductive cable comprises a plurality of electrically conductive strands.

4. A neurostimulation lead according to claim 3, wherein each electrically conductive wire comprises a plurality of electrically conductive strands.

5. A neurostimulation lead according to claim 4, wherein each strand comprises a stainless steel tube.

6. A neurostimulation lead according to claim 3, wherein each strand comprises a stainless steel tube.

7. A neurostimulation load according to claim 6, wherein each cable comprises more than ten strands.

8. A neurostimulation lead according to claim 7, wherein each cable comprises nineteen strands.

9. A neurostimulation lead according to claim 1, wherein the at least one lead retention element comprises a distal tine set disposed closer to the distal end than to the proximal end.

10. A neurostimulation lead according to claim 9, wherein the distal tine set comprises a plurality of elongate tines, each extending radially outward to a free end.

11. A neurostimulation lead according to claim 10, further comprising a plurality of paddle tines, each extending substantially parallel to at least one elongate tine.

12. A neurostimulation lead. according to claim 9, wherein the at least one lead retention element further comprises a proximal tine set disposed between the distal tine set and the proximal end.

13. A neurostimulation lead according to claim 12, wherein the distal tine set is located closer to the electrode than to the proximal tine set.

14. A neurostimulation lead according to claim 12, wherein both the proximal tine set and the distal tine set are located closer to the distal end than the proximal end.

15. A neurostimulation lead according to claim 12, wherein the proximal tine set comprises a plurality of caddie tines extending each extending radially outward to a proximal paddle free end.
